(19) United States
(12) Patent Application Publication
(10) Pub. No.: US 2024/0387045 A1
(43) Pub. Date: Nov. 21, 2024

(54) SYSTEMS AND METHODS FOR PROCESSING ELECTRONIC IMAGES TO QUANTIFY CORONARY MICROVASCUALAR DISEASE

(71) Applicant: HeartFlow, Inc., Mountain View, CA (US)

(72) Inventors: Sabrina LYNCH, Cupertino, CA (US); Michiel SCHAAP, Oegstgeest (NL); Adam UPDEPAC, Alamo, CA (US); Charles A. TAYLOR, Atherton, CA (US); Matthew SINCLAIR, London (GB); Nan XIAO, San Jose, CA (US); Souma SENGUPTA, Cupertino, CA (US)

(21) Appl. No.: 18/666,119

(22) Filed: May 16, 2024

Related U.S. Application Data
(60) Provisional application No. 63/502,756, filed on May 17, 2023.

(51) Int. Cl. G16H 50/20; A61P 9/10; G06V 10/44; G16H 30/20; G16H 30/40

(52) U.S. Cl. CPC G16H 50/20; A61P 9/10; G06V 10/44; G16H 30/20; G16H 30/40

(57) ABSTRACT

A computer-implemented method for processing electronic images to quantify coronary microvascular disease may include receiving imaging data of one or more captured electronic images. A first set of the imaging data may have been captured prior to an administration of one or more pharmacological agents, and a second set of the imaging data may have been captured subsequently. The method may further include providing the imaging data and a set of patient data to a machine-learning model. The machine-learning model may have been trained to identify coronary microvascular disease (CMD) features within the captured imaging data and the set of patient data and output one or more CMD measures and/or a predicted CMD endotype. The method may further include transmitting, to a user device, the one or more CMD measures and/or the predicted CMD endotype.

### CROSS-REFERENCE TO RELATED APPLICATION[S]

This application claims priority to U.S. Provisional Application No. 63/502,756, filed May 17, 2023, which is incorporated herein by reference in its entirety.

### TECHNICAL FIELD

Embodiments include systems and methods for image processing and, more particularly, machine-learning based techniques for quantifying coronary microvascular disease.

### BACKGROUND

Medical imaging may collect measurements and produce images of subject anatomy reconstructed from the measurements. The images may be processed and used by downstream tasks. Such downstream tasks may include, for example, analysis and/or interpretation. Various invasive metrics exist and have been used to investigate and diagnose coronary microvascular disease (CMD). The information gathered from these studies may generally help in the diagnosis and treatment planning for patients with microvascular disease, though these methods may be limited. Therefore, a need exists for determining and characterizing CMD and associated conditions.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, systems and methods are disclosed for processing electronic images to quantify or otherwise characterize coronary microvascular disease.

In an example, a computer-implemented method for processing electronic images to quantify coronary microvascular disease may include receiving imaging data of one or more captured electronic images. A first set of the imaging data may have been captured prior to an administration of one or more pharmacological agents to an imaged subject, and a second set of the imaging data may have been captured subsequent to the administration of the one or more pharmacological agents to the imaged subject. The method may further include providing the imaging data and a set of patient data to a machine-learning model. The machine-learning model may have been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype. The method may further include transmitting, to a user device, the one or more CMD measures and/or the predicted CMD endotype.

In another example, an image processing system for processing electronic images to quantify coronary microvascular disease may comprise a data storage device storing instructions for processing the electronic images, and a processor configured to execute the instructions to perform operations. The operations may include receiving imaging data of one or more captured electronic images. A first set of the imaging data may have been captured prior to an administration of one or more pharmacological agents to an imaged subject, and a second set of the imaging data may have been captured subsequent to the administration of the one or more pharmacological agents to the imaged subject. The operations may further include providing the imaging data and a set of patient data to a machine-learning model. The machine-learning model may have been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype. The operations may further include transmitting, to a user device, the one or more CMD measures and/or the predicted CMD endotype.

In a further example, a non-transitory computer-readable medium storing instructions that, when executed by one or more processors of an image processing system, may cause the one or more processors to perform a computer-implemented method for processing electronic images to quantify coronary microvascular disease. The method may include receiving imaging data of one or more captured electronic images. A first set of the imaging data may have been captured prior to an administration of one or more pharmacological agents to an imaged subject, and a second set of the imaging data may have been captured subsequent to the administration of the one or more pharmacological agents to the imaged subject. The method may further include providing the imaging data and a set of patient data to a machine-learning model. The machine-learning model may have been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype. The method may further include transmitting, to a user device, the one or more CMD measures and/or the predicted CMD endotype.

Additional objects and advantages of the techniques presented herein will be set forth in part in the description that follows, and in part will be apparent from the description, or may be learned by practice of the techniques presented herein. The objects and advantages of the techniques presented herein will be realized and attained by means of the elements and combinations particularly pointed out in the appended claims.

It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute part of this specification, illustrate various exemplary embodiments and together with the description, serve to explain the principles of the disclosed embodiments.

FIG. 1 depicts an exemplary environment for performing the techniques described herein.

FIG. 2 depicts an exemplary environment for processing electronic images to quantify coronary microvascular disease.

FIG. 3 depicts a flow chart of an exemplary method for processing electronic images to quantify coronary microvascular disease, according to one or more embodiments.

FIG. 4 depicts a flow chart of an exemplary method for predicting a microvascular resistance reserve, according to one or more embodiments.

FIG. 5 depicts a flow chart of an exemplary method for predicting a presence of microvascular disease, according to one or more embodiments.

FIG. 6 depicts a flow chart of an exemplary method determining one or more measures of coronary microvascular disease, according to one or more embodiments.

FIG. 7 depicts a flow chart of an exemplary method for estimating one or more pressure ratio metrics, according to one or more embodiments.

FIG. 8 depicts a flow diagram for training a machine-learning model, according to one or more embodiments.

FIG. 9 depicts an exemplary system that may execute techniques presented herein.

### DETAILED DESCRIPTION

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as, “substantially” and “generally,” are used to indicate a possible variation of ±10% of a stated or understood value.

Coronary arterial circulation provides blood supply to the muscle of the heart. The coronary arterial system originates from the root of the aorta in the left and right coronary artery circulations where large, epicardial arteries cover the surface of the heart and branch into smaller arteries that ultimately penetrate the muscle of the heart. These small arteries branch down to smaller vessels, the arterioles, which then terminate in the capillary vessels. Thus, the coronary arterial tree is often divided into the epicardial coronary arteries and the microvasculature. Blood vessels are not passive tubes, but rather they alter their caliber in response to changing physical forces and biochemical factors. This adaptive nature enables the heart to meet the varying demands of the body, ranging from a resting state to one of maximum exertion. However, in cases of progressive heart disease, the demand placed upon the muscle of the heart for blood circulation may not be as readily met, and the patient with progressive heart disease may therefore experience symptoms such as chest pain, reduced exercise capacity, and reduced quality of life.

Coronary microvascular disease (CMD) refers to the subset of disorders affecting the structure and function of coronary microcirculation and can be divided into structural and functional endotypes. In particular, vasodilation of the microvessels may be impaired in patients suffering from CMD. Conditions including atherosclerosis, hypertension, diabetes, pregnancy complications and preeclampsia may become risk factors in cases of CMD.

Because of the size of the microvasculature and the difficulty in directly observing the small vessels using medical imaging, it has conventionally been difficult to diagnose CMD using noninvasive methods. Various invasive methods exist and have been used to investigate and diagnose CMD. The information gathered from these methods may facilitate the diagnosis and treatment planning for patients with microvascular disease. Similarly, metrics derived from perfusion imaging modalities have also been proposed to diagnose CMD. However, interpreting such modalities is confounded by presence of epicardial disease, rendering it particularly challenging to diagnose CMD using non-invasive methods, including perfusion imaging. Due to the current diagnostic pathway, these measures are typically not implemented until after epicardial disease is ruled out.

Systems and methods related to predicting, detecting, and classifying the various endotypes of CMD with computed tomography angiography (CTA) are described in further detail below. The present system and methods may facilitate identification of microvascular disease and/or reduced vasodilatory capacity. The disclosed systems and methods improve the technology area of diagnosing and characterizing MVD. In particular, the disclosed systems and methods relate to non-invasive diagnosis, characterization, and/or quantification of MVD. For example, simultaneous non-invasive assessment of CAD and CMD may provide the opportunity for a more comprehensive evaluation of heart disease, tailored treatment options, and improved quality of life. The prevalence of CMD may be higher than previously thought and may be associated with worse clinical outcomes. The categorization of CMD into its various endotypes, including structural and functional CMD, may be necessary for proper diagnosis and treatment.

Additionally, because the microvasculature represents the majority of the coronary vascular system, the microvasculature may have a significant impact on the blood flow through the epicardial tree. Therefore, in non-invasively classifying the microvascular resistance, hyperemic blood flow may be more easily predicted, as well as pressure and resistance in the coronary epicardial vessels which may lead to improvements in the non-invasive computation of Fractional Flow Reserve (FFRct). In addition, by non-invasively classifying microvascular resistance, post-treatment FFRct may be predicted with more accuracy, and prediction of which patients will benefit from invasive treatment (e.g., percutaneous coronary intervention (PCI)), versus those that would not, may be improved. Thus, the disclosed systems and methods also improve the technology of patient-specific modeling and determination of FFRct.

The present system and methods provide, for example, for the quantification of the vasodilatory capacity of the epicardial coronary arteries from CTA scans before and after the administration of a pharmacologic agent and applying this information to non-invasively estimate microvascular function, its associated measures (e.g., microvascular resistance reserve (MRR), relative resistance reserve (RRR), microvascular resistance (MVR), index of microvascular resistance (IMR), measuring epicardial lesion severity, and coronary flow reserve (CFR), or hyperemic Microvascular Resistance (hMR)), and/or endotypes (e.g. structural and function CMD). Further, the present system and methods provide for using predicted patient-specific resting and hyperemic flows, pressures, resistances, and/or dilation capacities to improve non-invasive patient-specific resting and hyperemic pressure ratio metrics (e.g., Pd/Pa, instantaneous wave-free ratio (iFR), FFRct, and the like).

For example, the present disclosure uses CTA imaging data, acquired both before and after the administration of one or more pharmacological agents, for the non-invasive prediction of otherwise invasive measures used for the assessment of CMD, such as flow, pressure, resistance at both rest and hyperemia, MRR, RRR, MVR, CFR, as well as surrogates of the Index of Microvascular Resistance (IMR), hMR, and the like. The disclosure also uses the CTA imaging data for the prediction of endotypes of CMD, such as structural CMD and functional CMD and the like. Furthermore, the disclosed systems and methods include use of predicted patient-specific resting and hyperemic pressures, flows, or resistances, in addition to dilation capacities, to enable improved non-invasive patient-specific resting or hyperemic pressure ratio metrics (e.g. Pd/Pa, iFR, FFRct, and the like).

As described in further detail below, the variables, such as those described above, may be derived using CTA data acquired before and/or after the use of pharmacological agents. All data described herein, and the like, may also be used as input into a predictive algorithm (e.g. a machine-learning model and/or algorithm) that may estimate measures of CMD and/or its endotypes. In some examples, as described below, the vasodilatory capacity of a patient's microcirculation may be estimated by quantifying volume changes of the epicardial arteries visible in a CT image prior to and after administration of nitroglycerin.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistic regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

While several of the examples herein reference certain types of machine-learning, it should be understood that techniques according to this disclosure may be adapted to any suitable type of machine-learning. It should also be understood that the examples above and below are illustrative only. The techniques and technologies of this disclosure may be adapted to any suitable activity.

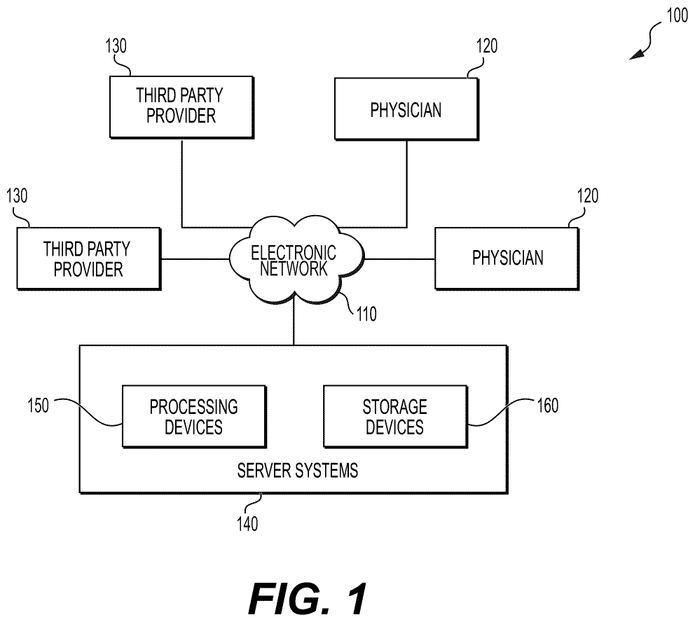

FIG. 1 depicts an example of an environment 100 in which a computer system may be implemented as server systems 140. In addition to server systems 140, the environment of FIG. 1 further includes a plurality of physicians 120 and third party providers 130, any of which may be connected to an electronic network 110, such as the Internet, through one or more computers, servers, and/or handheld mobile devices. In FIG. 1, physicians 120 and third party providers 130 may each represent a computer system, as well as an organization that uses such a system. For example, a physician 120 may be a hospital or a computer system of a hospital.

Physicians 120 and/or third party providers 130 may create or otherwise obtain medical images, such as images of the cardiac, vascular, and/or organ systems, of one or more patients. Physicians 120 and/or third party providers 130 may also obtain any combination of patient-specific information, such as age, medical history, blood pressure, blood viscosity, genetic risk factors, and other types of patient-specific information. Physicians 120 and/or third party providers 130 may transmit the patient-specific information to server systems 140 over the electronic network 110.

Server systems 140 may include one or more storage devices 160 for storing images and data received from physicians 120 and/or third party providers 130. The storage devices 160 may be considered to be components of the memory of the server systems 140. Server systems 140 may also include one or more processing devices 150 for processing images and data stored in the storage devices and for performing any computer-implementable process described in this disclosure. Each of the processing devices 150 may be a processor or a device that include at least one processor.

In some embodiments, server systems 140 may comprise and/or utilize a cloud computing platform with scalable resources for computations and/or data storage, and may run an application for performing methods described in this disclosure on the cloud computing platform. In such embodiments, any outputs may be transmitted to another computer system, such as a personal computer, for display and/or storage.

Other examples of computer systems for performing methods of this disclosure include desktop computers, laptop computers, and mobile computing devices such as tablets and smartphones.

A computer system, such as server systems 140, may include one or more computing devices. If the one or more processors of the computer system is implemented as a plurality of processors, the plurality of processors may be included in a single computing device or distribute among a plurality of computing devices. If a computer system comprises a plurality of computing devices, the memory of the computer system may include the respective memory of each computing device of the plurality of computing devices.

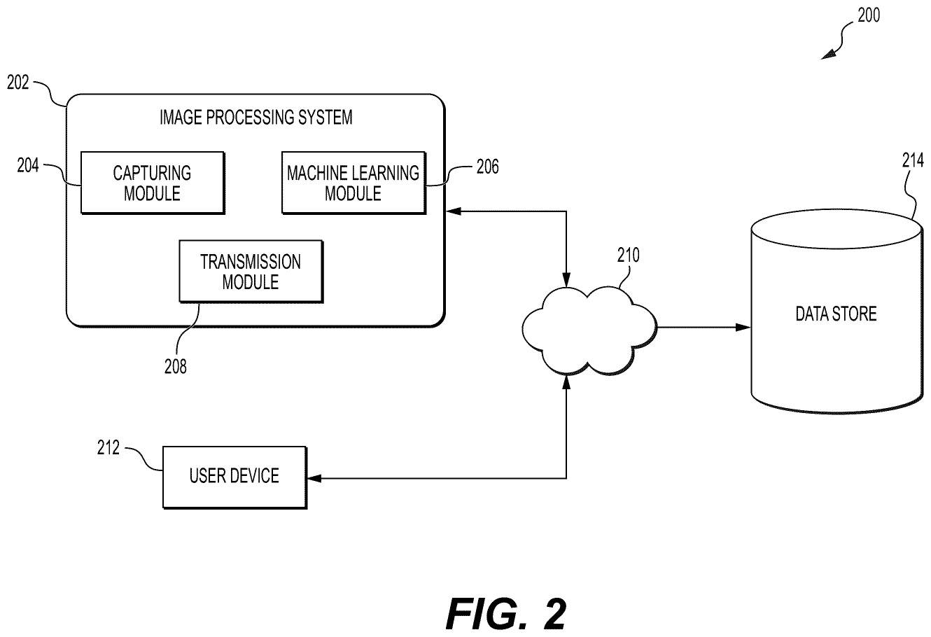

FIG. 2 depicts an exemplary environment 200 for processing electronic images to quantify coronary microvascular disease. One or more user device(s) 212 may communicate across an electronic network 210. The one or more user device(s) 212 may be associated with a user, e.g., a physician, an administrator of one or more components of environment 200, and/or the like. As will be discussed in further detail below, one or more image processing system(s) 202 may communicate with one or more of the other components of the environment 200 across electronic network 210.

The user device(s) 212 may be configured to enable a user to access and/or interact with other systems in the environment 200. For example, the user device(s) 212 may each be a computer system such as, for example, a desktop computer, a mobile device, a tablet, etc. In some embodiments, the user device(s) 212 may include one or more electronic application(s), e.g., a program, plugin, browser extension, etc., installed on a memory of the user device(s) 212. In some embodiments, the electronic application(s) may be associated with one or more of the other components in the environment 200. For example, the electronic application(s) may include one or more of system control software, system monitoring software, software development tools, etc.

In various embodiments, the environment 200 may include a data store 214 (e.g., database). The data store 214 may include a server system and/or a data storage system such as computer-readable memory such as a hard drive, flash drive, disk, etc. In some embodiments, the data store 214 includes and/or interacts with an application programming interface for exchanging data to other systems, e.g., one or more of the other components of the environment. The data store 214 may include and/or act as a repository or source for storing imaging data, output of the image processing system 200, output of the machine-learning model, and the like.

In some embodiments, the components of the environment 200 are associated with a common entity, e.g., a service provider, an account provider, a healthcare system, or the like. For example, in some embodiments, image processing system 202 and data store 214 may be associated with a common entity. In some embodiments, one or more of the components of the environment is associated with a different entity than another. For example, image processing system 202 may be associated with a first entity (e.g., a service provider) while data store 214 may be associated with a second entity (e.g., a storage entity providing storage services to the first entity). The systems and devices of the environment 200 may communicate in any arrangement. As will be discussed herein, systems and/or devices of the environment 200 may communicate in order to one or more of generate, train, or use a machine-learning model to process imaging data, among other activities.

As discussed in further detail below, the image processing system(s) 202 may one or more of (i) generate, store, train, communicate with, or use a machine-learning model configured to process imaging data and output one or more CMD measures, a predicted CMD endotype, and/or other relevant characteristics of a CMD diagnosis or evaluation. The image processing system(s) 202 may include a machine-learning model and/or instructions associated with the machine-learning model, e.g., instructions for generating a machine-learning model, training the machine-learning model, using the machine-learning model etc. The image processing system(s) 202 may include instructions for retrieving data, adjusting data, e.g., based on the output of the machine-learning model, and/or operating a display of the user device(s) 212 to output the one or more CMD measures and a predicted CMD endotype, e.g., as adjusted based on the machine-learning model. The image processing system(s) 202 may include training data, e.g., imaging data, and may include ground truth, e.g., (i) training imagining data and (ii) training patient data to output the one or more CMD measures, a predicted CMD endotype, and/or other relevant characteristics of a CMD diagnosis or evaluation.

As depicted in FIG. 2, image processing system(s) 202 may include capturing module 204. In various embodiments, capturing module 204 is configured to receive captured imaging data of one or more electronic images. In examples, the electronic images may captured using medical imaging techniques, such as by computed tomography angiography (CTA), coronary CT angiography (CCTA), and the like. In various implementations, a first set of the captured imaging data may be captured prior to an administration of one or more pharmacological agents to an imaged subject (e.g., a medical patient). In examples, the pharmacological agent may include a contrast agent, a vasodilator (e.g., nitroglycerin or adenosine), or a hyperemic agent (e.g. adenosine). A second set of the captured imaging data may be captured subsequent to the administration of the one or more pharmacological agents to the imaged subject.

As depicted in FIG. 2, image processing system(s) 202 may also include machine-learning module 206. In some embodiments, a system or device other than the image processing system(s) 202 is used to generate and/or train the machine-learning model. For example, such a system may include instructions for generating the machine-learning model, the training data and ground truth, and/or instructions for training the machine-learning model. A resulting trained-machine-learning model may then be provided to the image processing system(s) 202.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between imaging data and patient data such that the trained machine-learning model is configured to output one or more CMD measures, a predicted CMD endotype, and/or other relevant characteristics of a CMD diagnosis or evaluation.

In various embodiments, the variables of a machine-learning model may be interrelated in any suitable arrangement in order to generate the output. For example, in some embodiments, the machine-learning model may include image processing architecture that is configured to identify, isolate, and/or extract features in imaging data. For example, the machine-learning model may include one or more convolutional neural network (“CNN”) configured to identify features in the imaging data, and may include further architecture, e.g., a connected layer, neural network, etc., configured to determine one or more CMD measures, a predicted CMD endotype, and/or other relevant characteristics of a CMD diagnosis or evaluation.

In some embodiments, the machine-learning model of the image processing system 202 may include a Recurrent Neural Network (“RNN”). Generally, RNNs are a class of feed-forward neural networks that may be well adapted to processing a sequence of inputs. In some embodiments, the machine-learning model may include a Long Short Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of imaging data as input, and generate an output including the one or more CMD measures, a predicted CMD endotype, and/or other relevant characteristics of a CMD diagnosis or evaluation.

As depicted in FIG. 2, environment 200 may also include transmission module 208. In various embodiments, transmission module 208 may be configured to transmit, to a user device (e.g., such as user device 212), the one or more CMD measures, the predicted CMD endotype, and/or other relevant characteristics of a CMD diagnosis or evaluation. In examples, the one or more CMD measures, the predicted CMD endotype, and/or other relevant characteristics of a CMD diagnosis or evaluation may be included with a report that is generated by image processing system 202 and transmitted to user device 212 via network 210. As depicted in FIG. 2, environment 200 may also include electronic network 210. In various embodiments, the electronic network 210 may be a wide area network (“WAN”), a local area network (“LAN”), personal area network (“PAN”), or the like. In some embodiments, electronic network 210 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

Although depicted as separate components in FIG. 2, it should be understood that a component or portion of a component in the environment 200 may, in some embodiments, be integrated with or incorporated into one or more other components. In another example, the image processing system 202 may be integrated in a data storage system. The data storage system may be configured to communicate and/or receive/send data across electronic network 210 to other components of environment 200. In some embodiments, operations or aspects of one or more of the components discussed above may be distributed amongst one or more other components. Any suitable arrangement and/or integration of the various systems and devices of the environment 200 may be used.

Further aspects of the machine-learning model and/or how it may be utilized to process image data are discussed in further detail in the methods below. In the following methods, various acts may be described as performed or executed by a component from FIG. 2, such as the image processing system 202, the user device 212, or components thereof. However, it should be understood that in various embodiments, various components of the environment 200 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

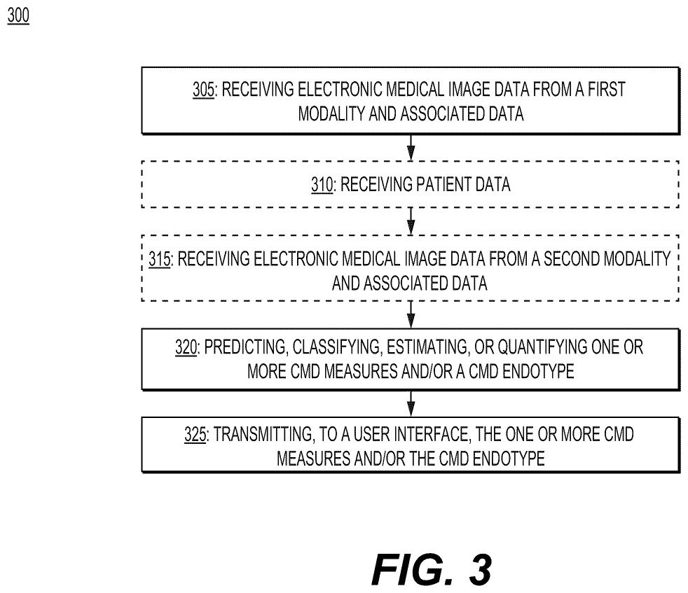

FIG. 3 depicts a flow chart of an exemplary method 300 for processing electronic images to quantify coronary microvascular disease. For example, method 300 may predict and classify CMD and/or its various endotypes non-invasively from (a) imaging data obtained before and/or after the administration of pharmacological agent(s) and/or (b) additional data. The steps of method 300 and their ordering are merely exemplary. Exemplary method 300 begins at step 305, wherein imaging data of one or more captured electronic images from a first imaging modality are received. The one or more electronic images may include one or more CTA or CCTA images of a medical patient. Although the term CTA is used below, it will be appreciated that such references also include CCTA. In examples, the electronic imaging data may be received by an image processing system, such as by capturing module 204 of image processing system 202, as depicted in FIG. 2. The electronic images may be images of coronary arteries, peripheral arteries, renal arteries, cerebral arteries, abdominal arteries, or other areas. The CTA may be single or multiphase.

Step 305 may also include receiving other data associated with the medical mages (e.g., electrocardiogram (ECG) data, scout images, and/or bolus tracking images). The data received may include derived data measures that may be extracted from CTA images/data (either only from a post-pharmacological agent (e.g., nitroglycerin or another nitrate) scan or from a pre-pharmacological agent scan. For example, derived data may include features derived from a difference between CTA scan(s) taken before administration of a pharmacological agent and after administration of a pharmacological agent, such as epicardial volume, vessel diameters, and or a volume-to-mass ratio). The derived data may further include plaque characterization, epicardial fat volume, a volume-to-mass ratio, features derived from myocardial intensities, contrast gradient-derived features in segmented coronary arteries, an/dor contrast differences among multiple CTA scans. Other examples of derived data include inflammation measures (e.g., pericoronary fat attenuation markers, such as fat attenuation index (FAI) and/fat radiomic profile (FRP)). Further examples of derived data include centerline properties including computed hemodynamics (e.g., FFRct, delta FFRct, wall shear stress, etc.), geometry information (e.g., diameter, area, bifurcation region, etc.), plaque information along centerlines, and/or the percentage of total coronary flow (or mass) that is supplied by a vessel (% Myo). It will be appreciated that references to receiving medical image data in the aspects below may also include receiving such other data.

In some examples, step 310 may include receiving CTA images obtained before the administration of a pharmacological agent and CTA images obtained after administration of a pharmacological agent. The pharmacological agent may include, for example, a vasodilator (e.g., nitroglycerin or another nitrate) or a hyperemia agent (e.g., adenosine). The pharmacological agents listed above are merely exemplary, and imaging data obtained before/after administration of other pharmacological agents is encompassed by this disclosure.

Optional step 310 may include receiving patient data. The patient data may include on-imaging data, such as, for example, atherosclerotic cardiovascular disease (ASCVD) or other risk factors, biomarkers, and/or DNA sequencing (genetic information). In examples, the patient data may include patient characteristics, such as medical history information (e.g., diabetes status), body mass index (BMI), gender, etc. The patient data may also include ECG data, such as an ECG signal. In aspects, the patient data may correspond to latent variables for a machine learning model.

In optional step 315, electronic image data from a second imaging modality may be received. The image data received in step 315 may be non-CTA imaging data. For example, the imaging modalities giving rise to the image data of step 315 may include perfusion imaging modalities, ultrasound, magnetic resonance (MR, which may include cardiovascular MR (CMR)), single photon emission computed tomography (SPECT), and/or positron emission tomography (PET). The imaging modality may also include higher-resolution CTA (e.g., photon counting CTA). As discussed above for step 305, the data received in step 315 may include data associated with the image data, such as derived data. For example, the data received in step 315 may include non-invasive ultrasound-based flow quantification or left ventricular (LV) deformation obtained from ultrasound or CMR imaging. The received in step 315 may have been previously obtained (i.e., past imaging data) or obtained contemporaneously or subsequently to the data of step 305.

Step 320 may include predicting, classifying, estimating, or quantifying one or more CMD measures and/or a CMD endotype. Step 320 may include using a computational method and/or a predictive method. A predictive method of step 320 may include using a machine-learning model. For example, step 320 may include providing the data received in steps 305, 310, and/or 315 to a machine-learning model. In various implementations, the machine-learning model may have been trained to identify coronary microvascular disease (CMD) features within the captured imaging data and the set of patient data and output one or more CMD measures and/or a predicted CMD endotype. In examples, the CMD features may include one or more identified differences between the first set of the captured imaging data and the second set of the captured imaging data. In aspects, the machine-learning model may use methods including logistic regression, random forests, XGBoost, and/or deep learning (e.g., tree-based long short-term memory (LSTM)). The models may use regression for continuous variables and classification for categorical variables. The machine-learning model may be executed by a machine-learning module, such as machine-learning module 206 of imaging processing system 202, as depicted in FIG. 2.

Step 320 may include characterizing (e.g., determining and/or predicting) various CMD measures. These measures may include, for example, rest blood flow (e.g., based on images obtained before administration of a hyperemic agent), hyperemic or stress blood flow (e.g., based on images obtained after administration of a hyperemic agent), pressure, and/or resistance, which may be indicative of a possible presence of CMD at a location. The measures may further include indices which may be derived from the above measures or from other measures, such as, for example, MRR, RRR, CFR, MVR, a surrogate of IMR, and/or a surrogate of hMR. Any of the above indices may be a continuous variable (regression) or a cut-off (classification) for CMD. Step 320 may include using the indices and/or measures for determining a presence or absence of CMD. Step 320 may also include determining a volume-to-mass ratio of the microcirculation at any location of the anatomy.

Step 320 may also include characterizing a CMD endotype. For example, step 320 may include differentiating between structural CMD and functional CMD. Structural CMD may result from, for example, arterial remodeling with intimal and/or medial wall thickening, perivascular fibrosis, and/or capillary rarefaction. Functional CMD may result from, for example, impaired coronary vasodilation due to endothelial and/or vascular smooth muscle cell dysfunction.

At step 320, the one or more CMD measures and the predicted CMD endotype may be transmitted to a user interface, such as to user device 212 via transmission module 208, as depicted in FIG. 2. The identified CMD features may include one or more identified differences between the first set of the captured imaging data and the second set of the captured imaging data. In such examples, a vasodilatory capacity may be determined based on the one or more identified differences. Further, a common vessel tree may be generated using the first set of the captured imaging data and the second set of the captured imaging data.

Methods 400, 500, and 600 are exemplary aspects implementing aspects of methods 300. For example, methods 400, 500, and 600 may differ according to a CMD measurement used to characterize a patient, the anatomies imaged, the additional input data used, and/or a characterization method employed. Methods 400, 500, and 600 are merely exemplary; method 300 includes combinations other than those described with respect to methods 400, 500, and 600.

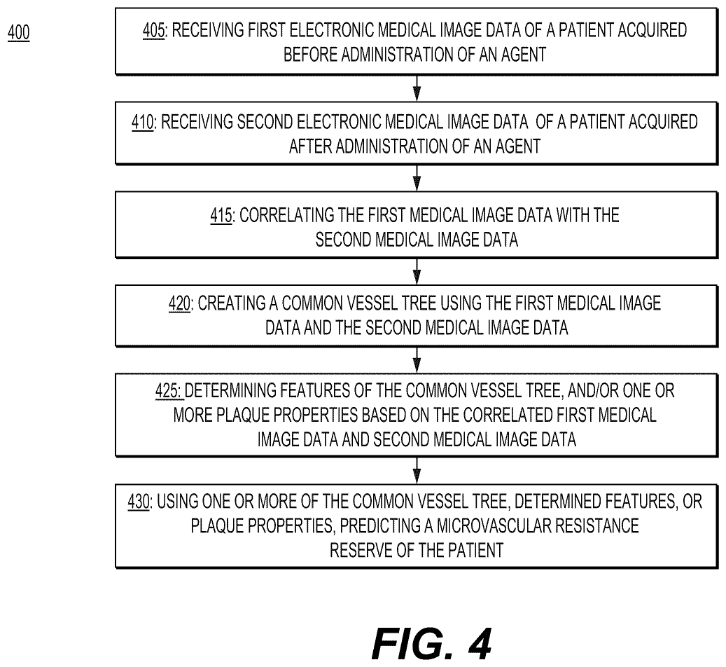

FIG. 4 depicts a flow chart of an exemplary method 400 for predicting a microvascular resistance reserve (MRR), which represents a ratio of a hypothetically healthy resistance of a coronary microvasculature at rest to a resistance of the coronary microvasculature at a stress (hyperemia) state. Method 400 utilizes a relationship between maximum vasodilation of epicardial vessels and a maximum amount of vasodilation of coronary microvasculature to estimate or predict MRR for a patient based on quantifying the amount of vasodilation of the coronary epicardial vessel due to administration of a pharmacological agent. Conventionally, MRR has been measured invasively. Thus, method 400 improves the technologies of patient-specific modeling and determination of MRR. The steps of method 400 and their ordering are merely exemplary.

At step 405, first electronic medical image data of a patient may be received (e.g., a first scan). In various embodiments, the first electronic medical image data may be acquired from the patient before the administration of a pharmacological agent. At step 410, second electronic medical image data of the patient may be received (e.g., a second scan). In various embodiments, the second electronic medical image data may be acquired after the administration of a pharmacological agent. In examples, the medical image data may be received using capturing module 204 of image processing system 202, as described with respect to FIG. 2. The images received in steps 405 and 410 may have any of the properties of images received in step 305 of method 300.

At step 415, the first medical image data received in step 405 is correlated with the second medical image data received in step 410. For example, the first electronic image data received in step 405 may be aligned with the second electronic image data received in step 410. For example, a first scan received in step 405 may be aligned with a second scan received in step 410. The alignment may result in a vessel tree of one of the first or second received medical image data (e.g., one of the scans, such as a first scan received in step 405). A deformation of each location and orientation of the vessel tree from the first or second received medical image data may be aligned to the anatomically corresponding location and orientation in the other of the first or second received image data (e.g., a second scan received in step 410).

At step 420, a common vessel tree may be created using the first medical image data and the second medical image data based on the correlation in step 415. In various embodiments, the aligned medical image data from step 415 may be used to create one common vessel tree. For example, a common vessel tree lumen may thereafter be extracted. At step 425, features of the common vessel tree and/or one or more plaque properties are determined based on the correlated first medical image data and second medical image data. For example, plaque morphological properties (e.g., a cross-sectional area), and/or plaque characterizations (e.g. plaque types) may be determined and/or extracted for one or more of the first received medical image data or the second received medical image data.

At step 430, a microvascular resistance reserve of the patient is predicted using the common vessel tree, the determined features, and/or plaque properties. In various embodiments, after alignment and feature extraction, the MRR may be predicted along the vessel tree using a machine-learning model trained to predict MRR based on the determined features and/or plaque properties. Step 430 may include using an amount of vasodilation of coronary epicardial vessels to estimate or predict MRR based on a relationship between a maximum vasodilaton of the epicardial vessels and a maximum amount of vasodilaton of the coronary microvasculature. In examples, a tree-based, long short-term memory network (LSTM), or Transformer model, that has been trained to propagate and aggregate the data (e.g., from leaves to root, or the like) may be used. In other examples, binning lumen and plaque features for a specific property (e.g., vessel size) may be performed. The binned data may then be used to predict MRR using multivariate regression. The predicted MRR may be used to determine whether the patient has CMD and, if so, an endotype of the CMD.

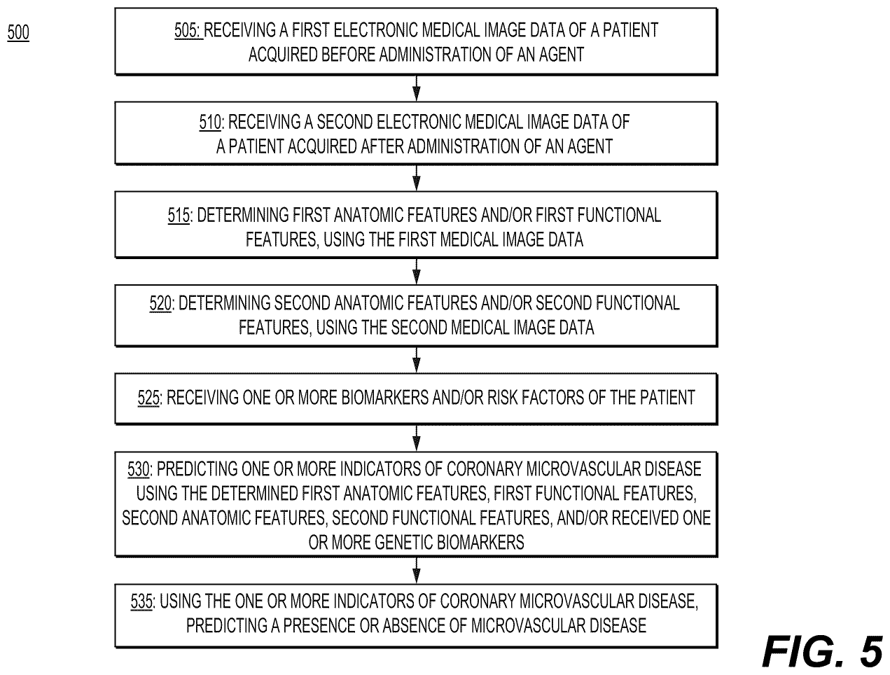

FIG. 5 depicts a flow chart of an exemplary method for predicting a presence of microvascular disease. The steps of method 500 and their ordering are merely exemplary. Method 500 utilizes a relationship between peripheral arterial disease and an incidence and/or severity of CMD. For example, the capacity of coronary microvasculature to vasodilate may be related to a presence, type, and/or distribution of peripheral arterial disease, biomarkers (e.g., LDL, HbA1C, BNP and the like), risk factors, symptoms, and/or genetics. Therefore, CTA may allow for visualization and analysis of peripheral artery disease including arterial plaque, stenosis, epicardial fat, as well as estimation of blood flow and arterial physiology. Thus, combining data from one or more CTAs, acquired after an administration of a pharmacological agent (e.g., a nitrate, or the like), together with risk factors and biomarkers, as well as a quantification of peripheral disease from CTA, may allow for the prediction of CMD. Method 500 thus improves the technology of characterizing CMD, among other things.

At step 505, first electronic medical image data of a patient may be received. In various embodiments, the first electronic medical image data may be acquired from the patient before the administration of a pharmacological agent. For example, the first electronic medical image data may be data of a full-body CTA scan. At step 510, second electronic medical image data of the patient may be received. In various embodiments, the second electronic medical image data may be acquired after the administration of a pharmacological agent. For example, the second electronic medical image data may be data of a full-body CTA scan. In examples, the medical image data may be received using capturing module 204 of image processing system 202, as described with respect to FIG. 2. In examples, two or more full body CTA scans of the peripheral arterial system may be acquired before and after the administration of a pharmacological agent such as nitroglycerine, or the like.

At step 515, first anatomic features and/or first functional features may be determined using the first medical image data. At step 520, second anatomical and/or second functional features may be determined using the second medical image data. In examples, such anatomic features and/or functional features determined in steps 515 and 520 may include arterial plaque, plaque morphology, stenosis, arterial fat, and resting flow and the like from the first and second medical image data (e.g., a first and second scan).

At step 525, one or more biomarkers of the patient may be received. For example, the biomarkers may be marks of one or more genetic variants associated with CMD. Additionally or alternatively, the biomarkers may include blood values, such as LDL value, HbA1C value, BNP value, etc. The biomarkers may also include risk factors (e.g., environmental or physical risk factors), and/or patient symptoms.

At step 530, one or more indicators of coronary microvascular disease may be predicted using the determined first anatomic features, first functional features, second anatomic features, second functional features, and/or received one or more genetic biomarkers. For example, the indicators may include any one of the measures described above with respect to step 320. Additionally or alternatively, the indicator may be a combination of any of the measures described with respect to step 320 and the received biomarkers and/or risk factors. The indicator may combine biomarkers and anatomic and/or functional features derived from the medical image data.

At step 535, a presence or absence of microvascular disease may be predicted using the one or more indicators of coronary microvascular disease. In various embodiments, the extracted information from the first and second medical image data may be combined and used to predict if a patient has CMD. The effects of multiple genetic variants associated with CMD may be combined with measures derived from CTA data. After extraction of the imaging and genetic risk features, these features may be combined with patient level risk factors and biomarkers, using a machine-learning model (e.g., an XBoost machine learning model), to predict an incidence of CMD at a territory and patient level. Multiple measures of CMD (e.g., dilation capacity, MRR, and/or CFR, or the like) may therefore be predicted in order to determine whether the patient has CMD.

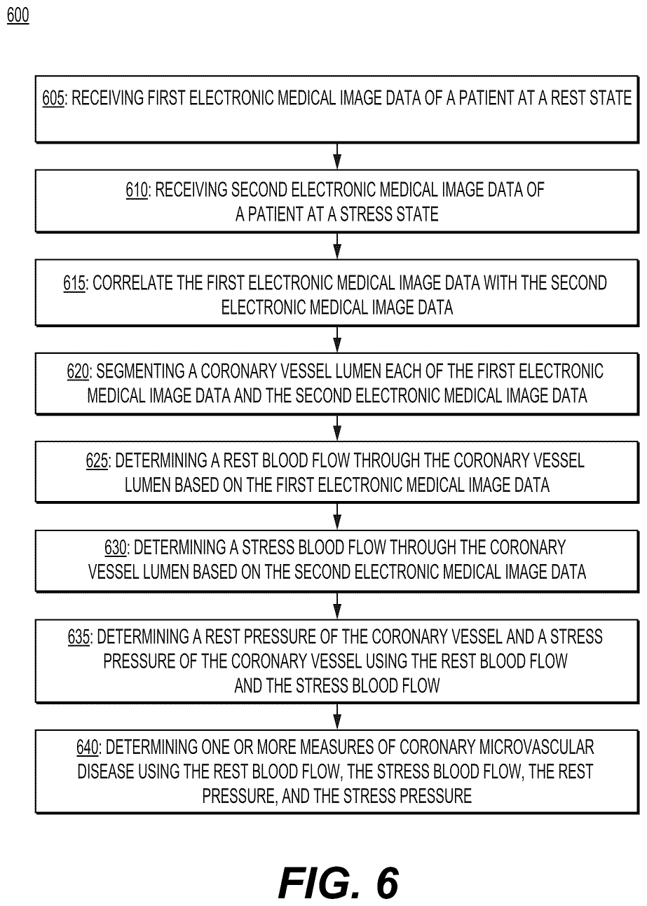

FIG. 6 depicts a flow chart of an exemplary method 600 for determining one or more measures of coronary microvascular disease. The steps of method 600 and their ordering are merely exemplary. Gradients in observed intensities of contrast measured along vessels imaged using CTA may contain information about the underlying blood flow at the time of acquisition. Specifically, analysis of the CTA image volume in conjunction with contrast bolus tracking images may allow estimation of vessel-specific flow rates at the time when the CTA volume was acquired. In examples, smaller changes in intensity along the vessel (e.g., lower values of the contrast gradient measured along the vessel, such as proximally to distally) may correspond to faster flow rates. In various embodiments, two coronary CTA acquisitions (e.g., pre- and post-administration of a pharmacological agent such as adenosine to induce hyperemia (stress)) may be performed. The contrast gradients from these two CCTA acquisitions may then be used to derive rest and stress flows in main coronary vessels. A simulation may then be generated to measure rest and stress flows to derive (e.g., non-invasively) the above-mentioned potential measures of CMD, including RRR, MRR, CFR, IMR, and the like. In examples, the simulation may be generated using artificial intelligence. Method 600 thus improves the technology of patient-specific modeling and characterization of CMD.

At step 605, first electronic medical image data of a patient may be received. In various embodiments, the first electronic medical image data may be acquired from the patient before the administration of a pharmacological agent. For example, the first medical image data may be data from a CTA scan. At step 610, second electronic medical image data of the patient may be received. In various embodiments, the second electronic medical image data may be acquired after the administration of a pharmacological agent, such as a hyperemic agent (e.g., adenosine). In examples, the medical image data may be received using capturing module 204 of image processing system 202, as described with respect to FIG. 2.

At step 615, the first electronic medial image data may be correlated with (e.g., aligned with) the second electronic medical image data. For example, a first CTA (e.g., CCTA) scan may be aligned with a second CTA (e.g., CCTA) scan. At step 620, a coronary vessel lumen of each of the first electronic medical image data and the second electronic medical image data may be segmented.

At step 625, a rest blood flow (e.g., flow rate) through the coronary vessel lumen may be determined based on the first electronic medical image data. For example, a contrast gradient may be used to determine a flow rate. In various embodiments, Transluminal Attenuation Flow Encoding (TAFE) and/or Advection Diffusion Flow Encoding (ADFE) may be used to translate contrast gradient(s) to flow rate(s). Translating contrast gradients to flow rates may rely on mathematical descriptions of the transport of the contrast material under the physiological flow conditions to estimate the flow rates given observations of the contrast concentrations. At step 630, a stress blood flow (e.g., flow rate) through the coronary vessel lumen may be determined based on the second electronic medical image data. Step 630 may utilize any of the techniques of step 625. In examples, the rest and stress flows may be derived from contrast gradients used as inputs in a simulation to determine a non-invasive metric of CMD.

At step 635, a rest pressure of the coronary vessel and a stress pressure of the coronary vessel may be determined using the rest blood flow and the stress blood flow.. In various embodiments, the pressures along the coronary vessels (at rest and at stress) may be derived using a fluid simulation (e.g., using techniques known in the art).

At step 640, one or more measures of coronary microvascular disease may be determined using the rest blood flow, the stress blood flow, the rest pressure, and the stress pressure. In examples, no other imaging modalities may be needed or required. In examples, measures for CMD that rely on combination(s) of rest blood flow, stress blood flow, rest pressure, and/or stress pressure may be used to derive one or more measures associated with characterizing CMD (e.g., RRR, MRR, CFR, IMR, and the like). The measures determined in step 640 may be used to characterize (e.g., diagnose) CMD and/or determine an endotype of CMD.

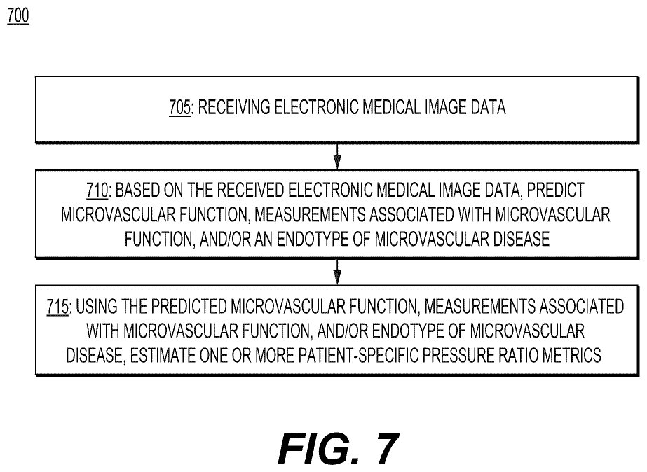

FIG. 7 depicts a flow chart of an exemplary method for estimating one or more pressure ratio metrics. The steps of method 700 and their ordering are merely exemplary. In various embodiments, method 700 may be used in conjunction with any of the other methods described herein. At step 705, electronic medical image data may be received. In examples, the medical image data may be received using capturing module 204 of image processing system 202, as described with respect to FIG. 2. The medical image data received in step 705 may include image data (e.g., CTA data) obtained before and after administration of a pharmacological agent, such as any of those discussed above.

Therefore, at step 710, microvascular function, measurements associated with microvascular function, and/or an endotype of microvascular disease may be predicted based on the received electronic medical image data. Step 710 may utilize any of the aspects of methods 300, 400, 500, 600 described above (e.g., the outputs of methods 300, 400, 500, and/or 600). In various embodiments, quantification of the vasodilatory capacity of the epicardial coronary arteries from CTA scans before and after the administration of a pharmacologic agent may enable a non-invasive determination of microvascular function, its associated measures (e.g., MRR, RRR, CFR, MVR, HMR, hMR, and the like), and endotypes (e.g., structural and functional CMD). In using the predicted patient-specific resting and hyperemic flows, pressures, resistances, and/or dilation capacities may improve non-invasive patient-specific resting and hyperemic pressure ratio metrics (e.g., Pd/Pa, iFR, FFRct, and the like).

At step 715, one or more patient-specific pressure ratio metrics may be estimated and/or predicted using the predicted microvascular function, measurements associated with microvascular function, and/or endotype of microvascular disease. For example, measures and/or other characteristics associated with CMD (e.g., outputs of methods 300, 400, 500, and/or 600) may be used to determine non-invasive pressure ratio metrics (e.g., Pd/Pa, iFR, FFRct, etc.). In some examples, predicated patient-specific resting and hyperemic/stress flows (e.g., determined in steps 625 and/or 630), pressures (e.g., determined in step 635), resistances, and/or dilation capacities (e.g. as determined in method 400) may be utilized to non-invasively determine one or more pressure ratio metric (e.g., resting and hyperemic pressure ratio metrics).

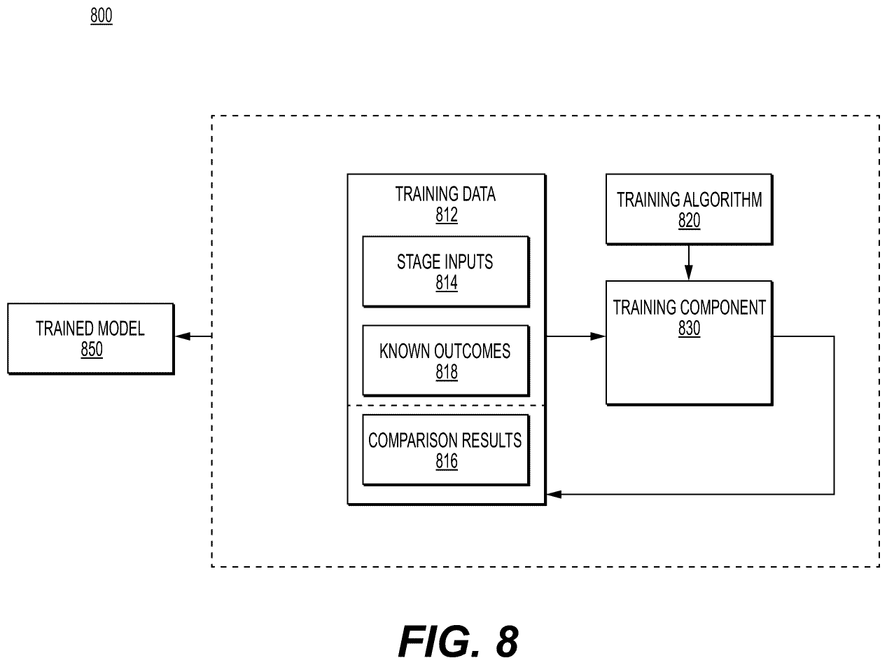

FIG. 8 depicts a flow diagram for training a machine-learning model. The trained machine learning model of FIG. 8 may be used in any of methods 300-700. As shown in flow diagram 800 of FIG. 8, training data 812 may include one or more of stage inputs 814 and known outcomes 818 related to a machine-learning model to be trained. The stage inputs 814 (e.g., medical imaging data such as CTA or CCTA images, on-imaging data, patient characteristics such as genetic factors, or ECG data, or the like) may be from any applicable source including a component or set shown in the figures provided herein. The known outcomes 818 (e.g., known CMD measures and the predicted CMD endotypes) may be included for machine-learning models generated based on supervised or semi-supervised training. An unsupervised machine-learning model might not be trained using known outcomes 818. Known outcomes 818 may include known or desired outputs for future inputs similar to or in the same category as stage inputs 814 that do not have corresponding known outputs.

The training data 812 and a training algorithm 820 may be provided to a training component 830 that may apply the training data 812 to the training algorithm 820 to generate a trained machine-learning model 850. According to an implementation, the training component 830 may be provided comparison results 816 that compare a previous output of the corresponding machine-learning model to apply the previous result to re-train the machine-learning model. The comparison results 816 may be used by the training component 830 to update the corresponding machine-learning model. The training algorithm 820 may utilize machine-learning networks and/or models including, but not limited to a deep learning network such as Deep Neural Networks (DNN), Convolutional Neural Networks (CNN), Fully Convolutional Networks (FCN), Transformers, and Recurrent Neural Networks (RCN), probabilistic models such as Bayesian Networks and Graphical Models, and/or discriminative models such as Decision Forests and maximum margin methods, or the like. The output of the flow diagram 800 may be a trained machine-learning model 850.

A machine-learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine-learning model (e.g., a trained model) based on the training. Once trained, the machine-learning model may output machine-learning model outputs in accordance with the subject matter disclosed herein. According to an implementation, one or more machine-learning models disclosed herein may continuously update based on feedback associated with use or implementation of the machine-learning model outputs.

It should be understood that aspects in this disclosure are exemplary only, and that other aspects may include various combinations of features from other aspects, as well as additional or fewer features.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

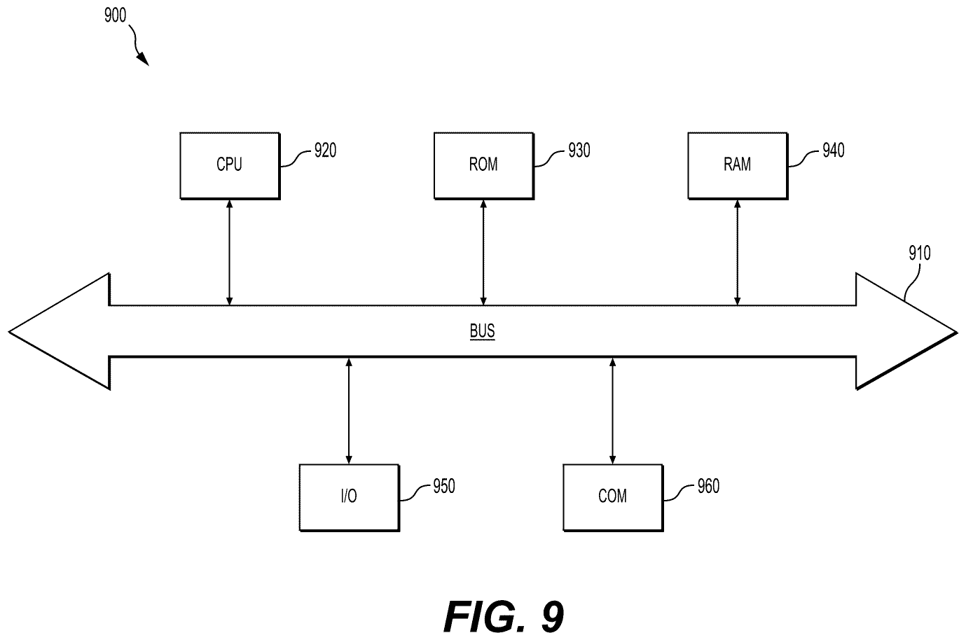

As shown in FIG. 9, device 900 may include a central processing unit (CPU) 920. CPU 920 may be any type of processor device including, for example, any type of special purpose or a general-purpose microprocessor device. As will be appreciated by persons skilled in the relevant art, CPU 920 also may be a single processor in a multi-core/multiprocessor system, such system operating alone, or in a cluster of computing devices operating in a cluster or server farm. CPU 920 may be connected to a data communication infrastructure 910, for example a bus, message queue, network, or multi-core message-passing scheme.

Device 900 may also include a main memory 940, for example, random access memory (RAM), and also may include a secondary memory 930. Secondary memory 930, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 930 may include similar means for allowing computer programs or other instructions to be loaded into device 900. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 900.

Device 900 also may include a communications interface (“COM”) 960. Communications interface 960 allows software and data to be transferred between device 900 and external devices. Communications interface 960 may include a modem, a network interface (such as an Ethernet card), a communications port, a PCMCIA slot and card, or the like. Software and data transferred via communications interface 960 may be in the form of signals, which may be electronic, electromagnetic, optical or other signals capable of being received by communications interface 960. These signals may be provided to communications interface 960 via a communications path of device 900, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 900 may also include input and output ports 950 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

One or more techniques presented herein may enable a user, to better interact with a digital image of a glass slide that may be presented on a screen, in a virtual reality environment, in an augmented reality environment, or via some other form of visual display. One or more techniques presented herein may enable a natural interaction closer to traditional microscopy with less fatigue than using a mouse, keyboard, and/or other similar standard computer input devices.

The controllers disclosed herein may be comfortable for a user to control. The controllers disclosed herein may be implemented anywhere that digital healthcare is practiced, namely in hospitals, clinics, labs, and satellite or home offices. Standard technology may facilitate connections between input devices and computers (USB ports, Bluetooth (wireless), etc.) and may include customer drivers and software for programming, calibrating, and allowing inputs from the device to be received properly by a computer and visualization software.

The foregoing general description is exemplary and explanatory only, and not restrictive of the disclosure. Other embodiments of the invention may be apparent to those skilled in the art from consideration of the specification and practice of the invention disclosed herein. It is intended that the specification and examples be considered as exemplary only.

Instructions executable by one or more processors may also be stored on a non-transitory computer-readable medium. Therefore, whenever a computer-implemented method is described in this disclosure, this disclosure shall also be understood as describing a non-transitory computer-readable medium storing instructions that, when executed by one or more processors of a computer system, configure and/or cause the one or more processors to perform the computer-implemented method. Examples of non-transitory computer-readable media include random-access memory (RAM), read-only memory (ROM), solid-state storage media (e.g., solid state drives), optical storage media (e.g., optical discs), and magnetic storage media (e.g., hard disk drives). A non-transitory computer-readable medium may be part of the memory of a computer system or separate from any computer system.

A computer system may include one or more computing devices. If a computer system includes a plurality of processors, the plurality of processors may be included in a single computing device or distributed among a plurality of computing devices. A processor may be a central processing unit (CPU), a graphics processing unit (GPU), or another type of processing unit. The term “computational device,” as used in this disclosure, is interchangeable with “computing device.” An “electronic storage device” may include any of the non-transitory computer-readable media described above.

What is claimed is:

1. A computer-implemented method for processing electronic images to quantify coronary microvascular disease, the method comprising:
receiving, by one or more processors of an image processing system, imaging data of one or more captured electronic images, wherein a first set of the imaging data was captured prior to an administration of one or more pharmacological agents to an imaged subject, and wherein a second set of the imaging data was captured subsequent to the administration of the one or more pharmacological agents to the imaged subject;
providing, by the one or more processors, the imaging data and a set of patient data to a machine-learning model, wherein the machine-learning model has been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype; and
transmitting, by the one or more processors and to a user device, the one or more CMD measures and/or the predicted CMD endotype.

2. The computer-implemented method of claim 1, further comprising:
determining, by the one or more processors, a microvascular resistance reserve (MRR) using the captured imaging data.

3. The computer-implemented method of claim 2, wherein the set of patient data includes the MRR.

4. The computer-implemented method of claim 1, wherein the identified CMD features include one or more identified differences between the first set of the captured imaging data and the second set of the captured imaging data.

5. The computer-implemented method of claim 4, further comprising:
determining, by the one or more processors, a vasodilatory capacity based on the one or more identified differences.

6. The computer-implemented method of claim 1, wherein the set of patient data includes one or more biomarkers of a patient.

7. The computer-implemented method of claim 1, wherein the one or more electronic images comprise one or more CT angiography (CCTA) images.

8. The computer-implemented method of claim 1, further comprising:
generating, by the one or more processors, a common vessel tree using the first set of the captured imaging data and the second set of the captured imaging data.

9. An image processing system for processing electronic images to quantify coronary microvascular disease, the system comprising:
a data storage device storing instructions for processing the electronic images; and
a processor configured to execute the instructions to perform operations comprising:
receiving, by one or more processors of an image processing system, imaging data of one or more captured electronic images, wherein a first set of the imaging data was captured prior to an administration of one or more pharmacological agents to an imaged subject, and wherein a second set of the imaging data was captured subsequent to the administration of the one or more pharmacological agents to the imaged subject;
providing, by the one or more processors, the imaging data and a set of patient data to a machine-learning model, wherein the machine-learning model has been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype; and
transmitting, by the one or more processors and to a user device, the one or more CMD measures and/or the predicted CMD endotype.

10. The image processing system of claim 9, the operations further comprising:
determining, by the processor, a microvascular resistance reserve (MRR) using the captured imaging data.

11. The image processing system of claim 10, wherein the set of patient data includes the MRR.

12. The image processing system of claim 9, wherein the identified CMD features include one or more identified differences between the first set of the captured imaging data and the second set of the captured imaging data.

13. The image processing system of claim 12, the operations further comprising:
determining, by the processor, a vasodilatory capacity based on the one or more identified differences.

14. The image processing system of claim 9, wherein the set of patient data includes one or more biomarkers of a patient.

15. The image processing system of claim 9, wherein the one or more electronic images comprise one or more CT angiography (CCTA) images.

16. The image processing system of claim 9, the operations further comprising:
generating, by the processor, a common vessel tree using the first set of the captured imaging data and the second set of the captured imaging data.

17. A non-transitory computer-readable medium storing instructions that, when executed by one or more processors of an image processing system, cause the one or more processors to perform a computer-implemented method for processing electronic images to quantify coronary microvascular disease, the method comprising:
receiving, by one or more processors of an image processing system, imaging data of one or more captured electronic images, wherein a first set of the imaging data was captured prior to an administration of one or more pharmacological agents to an imaged subject, and wherein a second set of the imaging data was captured subsequent to the administration of the one or more pharmacological agents to the imaged subject;
providing, by the one or more processors, the imaging data and a set of patient data to a machine-learning model, wherein the machine-learning model has been trained, using one or more gathered and/or simulated sets of imaging data and one or more gathered and/or simulated sets of patient data, to identify coronary microvascular disease (CMD) features within the captured imaging data and the set of patient data and output one or more CMD measures and/or a predicted CMD endotype; and
transmitting, by the one or more processors and to a user device, the one or more CMD measures and/or the predicted CMD endotype.

18. The non-transitory computer-readable medium of claim 17, wherein the method further comprises:
determining, by the one or more processors, a microvascular resistance reserve (MRR) using the captured imaging data.

19. The non-transitory computer-readable medium of claim 18, wherein the set of patient data includes the MRR.

20. The non-transitory computer-readable medium of claim 17, wherein the identified CMD features include one or more identified differences between the first set of the captured imaging data and the second set of the captured imaging data.
